(19) United States
(12) Patent Application Publication
(10) Pub. No.: US 2022/0387224 A1
(43) Pub. Date: Dec. 8, 2022

(54) OXYGENATED HEMOGLOBIN AND APPARATUSES, SYSTEMS, AND METHODS THEREFORE

(71) Applicant: KCI Licensing, Inc., San Antonio, TX (US)

(72) Inventors: Justin Alexander LONG, Lago Vista, TX (US); Christopher Brian LOCKE, Bournemouth (GB); Kathleen L. DERRICK, San Antonio, TX (US)

(21) Appl. No.: 17/774,308

(22) PCT Filed: Nov. 6, 2020
(86) PCT No.: PCT/IB2020/060488

§ 371 Date: May 4, 2022

Related U.S. Application Data
(60) Provisional application No. 62/932,003, filed on Nov. 7, 2019.

(51) Int. Cl. A61F 13/02

(52) U.S. Cl. CPC A61F 13/0206; A61M 2202/0433; A61M 2202/0208

(57) ABSTRACT

This disclosure describes devices, systems, and methods related to oxygenated hemoglobin, the generation thereof, and the use thereof. An exemplary oxygenated hemoglobin therapy system includes an oxygen source configured to provide oxygen and a hemoglobin source configured to provide topical hemoglobin. The therapy system may also include a mixer which has a first inlet, a second inlet, and an outlet. The mixer is configured to mix the oxygen and the topical hemoglobin to form a mixture and to provide the mixture to a dressing via the outlet. The therapy system may further include the dressing. The oxygenated hemoglobin therapy systems described herein are suitable for use in medical devices, such as bandages, drapes, dressings, and wound closures.

### CROSS-REFERENCE TO RELATED APPLICATIONS

This application claims the benefit of priority to U.S. Provisional Application No. 62/932,003, filed on Nov. 7, 2019, which is incorporated herein by reference in its entirety.

### TECHNICAL FIELD

Aspects of the present disclosure relate generally to oxygen therapy and hemoglobin, such as oxygen therapy with aerosol hemoglobin for use with medical devices, and more specifically, but not by way of limitation, to an apparatus for mixing oxygen and hemoglobin and an apparatus, system, and method for treating a wound with oxygenated hemoglobin.

### BACKGROUND

Clinical studies and practice have shown that oxygen therapy in proximity to a tissue site can augment and accelerate growth of new tissue at the tissue site. Applications of this phenomenon are diverse, but have proven particularly advantageous for treating wounds. Regardless of the cause of a wound, whether trauma, surgery, etc., care of the wound can impact the outcome. Treatment of wounds with oxygen therapy at pressures above ambient may be commonly referred to as “hyperbaric oxygen wound therapy,” but is also known by other names, including “hyperbaric oxygen therapy,” “positive-pressure therapy,” “positive pressure wound therapy,” and “hyperbaric therapy,” as illustrative, non-limiting examples. Oxygen therapy may provide one or more benefits, including migration of epithelial and subcutaneous tissues, improved blood flow, reduced infection, and/or micro-deformation of tissue at a wound site. These benefits can increase development of granulation tissue and reduce healing times.

Oxygen therapy is typically provided by a positive-pressure system (e.g., a pressurized system or a hyperbaric system) including one or more components and/or one or more devices. To illustrate, a conventional positive-pressure system may include a dressing, one or more tubes, a therapy device (e.g., a positive-pressure source), or a combination thereof, as illustrative, non-limiting examples. The dressing may be placed at a tissue site and coupled to the positive-pressure source via the tube. The positive-pressure source is configured to establish positive-pressure (e.g., a hyperbaric environment) at the dressing and the dressing is configured to maintain the positive-pressure at the tissue site.

However, excess positive-pressure at the dressing and tissue site can restrict or close capillaries, which reduces blood flow and may increases healing times. Additionally, such benefits of pressurized oxygen are only present when the wound is subject to positive pressure and oxygen. Thus, a pressure loss or lack of oxygen reduces or eliminates the benefits and it takes time to restore the pressure and/or oxygen when changing components or after a leak is remedied, which decreases efficacy.

While providing oxygen to a wound is beneficial, the human body is not very efficient at processing oxygen this way. Oxygen is typically absorbed by red blood cells in the lungs and oxygen is not usually absorbed through the skin or wounds in large quantities. Thus, although oxygen is beneficial for healing, oxygen is not efficiently absorbed by wounds and red blood cells thereof. Therefore, while conventional oxygen therapies are effective, conventional oxygen therapies suffer from exploiting an inefficient process to absorb oxygen.

### SUMMARY

This disclosure describes oxygenated hemoglobin, apparatuses (e.g., delivery device) including oxygenated hemoglobin, and systems and methods related to forming and/or using oxygenated hemoglobin. The oxygenated hemoglobin described herein includes hemoglobin (e.g., aerosol hemoglobin) which has been mixed with and/or saturated with oxygen. The oxygenated hemoglobin provides oxygen to a wound more efficiently then gaseous oxygen and may be used in pressurized and/or oxygen therapy, such as wound therapy. Accordingly, the oxygenated hemoglobin systems described herein are more efficient and promote improved healing and reduced recovery times.

In some implementations, oxygenated hemoglobin is provided by a therapy system. An exemplary therapy system may include an oxygen source configured to provide oxygen and a hemoglobin source configured to provide topical hemoglobin. The therapy system may also include a mixer which has a first inlet, a second inlet, and an outlet. The mixer is configured to mix the oxygen and the topical hemoglobin to form a mixture and to provide the mixture to a dressing via the outlet. The therapy system may further include the dressing. The oxygenated hemoglobin therapy systems described herein are suitable for use in medical devices, such as bandages, drapes, dressings, and wound closures.

Thus, the oxygenated hemoglobin therapy systems of the present disclosure are configured to provide oxygen, which is bound to hemoglobin, to a wound site. Hemoglobin (Hb) is the primary vehicle for transporting oxygen in the blood. Accordingly, the hemoglobin and oxygen mixture may be absorbed by the wound, such as red blood cells thereof, more efficiently and more easily as the mixture takes advantage of how the body normally transports/transfers oxygen. Therefore, the oxygenated hemoglobin therapy systems enable improved wound care and therapy and reduced healing times, thereby advancing patient comfort and confidence in the treatment.

In some implementations, the mixing device of the oxygenated hemoglobin therapy system is separate from the dressing. For example, the mixing device may be a separate, discrete device or may be incorporated into another component, such as a therapy device. In some such implementations, the oxygen and/or the hemoglobin may be incorporated into the therapy device. In other implementations, the mixing device of the oxygenated hemoglobin therapy system is incorporated or integrated with the dressing. For example, the dressing receives oxygen and hemoglobin and mixes the two at the dressing and/or wound site.

The mixing devices of the present disclosure are configured to mix oxygen and hemoglobin to form a mixture and include different types of mixers. As illustrative, non-limiting examples, the mixing device may include nozzle mixers, swirler mixers, and porous mixers, or a combination thereof. Alternatively, some oxygenated hemoglobin therapy systems may not include a mixing device. To illustrate, the oxygen and hemoglobin may come premixed in such implementations, and the pre-combined mixture is provided to the dressing.

In some embodiments of the present systems (e.g., a therapy system), the present systems comprise: an oxygen source configured to provide oxygen; a hemoglobin source configured to provide topical hemoglobin; a mixer including a first inlet, a second inlet, and an outlet, the mixer configured to mix the oxygen and the topical hemoglobin to form a mixture and to provide the mixture to a dressing via the outlet; and the dressing.

In some of the foregoing embodiments of the present systems, the oxygen source is a hydrolysis device configured to generate the oxygen, and wherein the topical hemoglobin is aerosol hemoglobin. Additionally, or alternatively, the topical hemoglobin is natural hemoglobin, synthetic hemoglobin, or a mixture thereof.

In some of the foregoing embodiments of the present systems, the topical hemoglobin is carbonylated hemoglobin. Additionally, or alternatively, the topical hemoglobin is fetal hemoglobin, adult hemoglobin, or a mixture thereof. In some of the foregoing embodiments of the present systems, the oxygen is medically pure humidified oxygen.

In some of the foregoing embodiments of the present systems, the mixer comprises one of: a spray nozzle to inject one of the oxygen or the topical hemoglobin into the other of the oxygen or the topical hemoglobin to form a homogenous mixture; an in-line mixer configured to generate a homogenous mixture; or a porous structure configured to mix the oxygen and the topical hemoglobin to form a homogenous mixture. Additionally, or alternatively, the present systems further comprise tubing and a controller configured to control delivery of the oxygen, the topical hemoglobin, the mixture, or a combination thereof, to the dressing. In some of the foregoing embodiments of the present systems, the present systems further comprise one or more sensors.

In some embodiments of the present wound dressings, the present wound dressings comprise: an absorbent material; and a mixer coupled to the absorbent material and including an oxygen inlet, a hemoglobin inlet, and an outlet, the mixer configured to mix oxygen and hemoglobin to form a mixture and to provide the mixture to the absorbent material via the outlet.

In some of the foregoing embodiments of the present wound dressings, the mixer comprises a spray nozzle configured to inject one of the oxygen or the topical hemoglobin into the other of the oxygen or the topical hemoglobin to form a homogenous mixture. Alternatively, the mixer comprises an in-line mixer configured to generate a homogenous mixture. In some such implementations, the in-line mixer has a spiral or square shape conduit for mixing the oxygen and the hemoglobin.

In some of the foregoing embodiments of the present wound dressings, the mixer comprises porous structure configured to mix the oxygen and the topical hemoglobin to form a homogenous mixture. In some such implementations, the porous structure comprises a porous polymeric exchange material.

In some of the foregoing embodiments of the present wound dressings, the present wound dressing further comprise a manifold coupled to the absorbent material and to the mixer. In some such implementations, the manifold comprises polyether based polyurethane foam, and wherein the mixer provides the mixture to the absorbent material via the manifold. Additionally, the absorbent material may comprise a printed absorbent.

In some of the foregoing embodiments of the present wound dressings, the present wound dressings further comprise a pad and a drape. Additionally, or alternatively, the present wound dressings further comprise a sensor.

In some embodiments of the present methods, the present methods comprise: mixing oxygen and topical hemoglobin to form a mixture; and applying the mixture to a wound dressing attached to a tissue site. In some of the foregoing embodiments of the present methods, applying the mixture corresponds to hyperbaric oxygen treatment. In some such implementations, the present methods further comprise applying negative pressure therapy after the hyperbaric oxygen treatment, wherein the hyperbaric oxygen treatment and the negative pressure therapy may be cycled.

In some of the foregoing embodiments of the present methods, the present methods further comprise generating the oxygen. Additionally, or alternatively, the present methods further comprise: providing the oxygen from an oxygen source; and providing the topical hemoglobin in aerosol form from a compressed hemoglobin source.

In some embodiments of the present methods, the present methods comprise providing pressurized wound therapy to a tissue site via a wound dressing; and while providing the pressurized wound therapy, providing a mixture of oxygen and topical hemoglobin to the wound dressing. In some of the foregoing embodiments of the present methods, the present methods further comprise: providing the oxygen from an oxygen source; providing the topical hemoglobin in aerosol form from a compressed hemoglobin source; and mixing the oxygen and the topical hemoglobin to form the mixture. Additionally, or alternatively, the present methods further comprise generating the oxygen.

In some embodiments of the present kits (e.g., a kit for oxygen wound therapy), the present kits comprise: one or more wound dressings as in claim 10. In some of the foregoing embodiments of the present kits, the kits further comprise an oxygen source, a hemoglobin source, a mixture of oxygen and hemoglobin, or a combination thereof.

In some embodiments of the present kits (e.g., a kit for oxygen wound therapy), the present kits comprise: an oxygen source configured to provide oxygen; a hemoglobin source configured to provide topical hemoglobin; a mixer including a first inlet, a second inlet, and an outlet, the mixer configured to mix the oxygen and the topical hemoglobin to form a mixture and to provide the mixture to a dressing via the outlet. In some of the foregoing embodiments of the present kits, the present kits further comprise one or more dressings.

In some embodiments of the present systems (e.g., a therapy systems), the present systems comprise: an oxygen source configured to provide oxygen; a hemoglobin source configured to provide topical hemoglobin; means for mixing the oxygen and the topical hemoglobin to form a mixture and to provide the mixture to a dressing via the outlet.

In some of the foregoing embodiments of the present systems, the oxygen, the topical hemoglobin, or both are stored under pressure. Additionally, or alternatively, the present systems further comprise: a dressing coupled to the means for mixing; and a pump coupled to the means for mixing and configured to transport the mixture to the dressing.

In some embodiments of the present systems (e.g., a therapy system), the present systems comprise: a wound dressing; and a container coupled to the wound dressing, the configured to provide a mixture of oxygen and hemoglobin to the dressing.

In some of the foregoing embodiments of the present systems, the container further comprising means for mixing oxygen and topical hemoglobin to form the mixture. Additionally, or alternatively, the container includes the mixture of oxygen and hemoglobin, and wherein the mixture is stored under pressure. In some of the foregoing embodiments of the present methods, the present systems, the present systems further comprise a pump coupled to the container and configured to transport the mixture to the dressing.

As used herein, the term “switchable” will be used to refer to adhesives which can be changed at least from one state or phase (e.g., a high tack and/or peel strength state) to another state or phase (e.g., a low tack and/or peel strength state, such as a non-tacky state). Recognizing that the expression “low tack and/or peel strength” is a relative term, it will be defined here as meaning a condition of a minimum reduction in tackiness which the adhesive reaches after switching from the high tack and/or peel strength state. The reduction in tack or peel force may be as great as 99% or as little as 30%. Typically, the reduction in tack or peel force is between 70% and 90%.

As used herein, the term “peel strength” will be used to refer to a strength of adhesives measured by a 180 degree peel test on stainless steel. Recognizing that a bond strength of adhesive depends on the medium to which it adheres and that tissue composition can vary greatly, the measured peel strength is indicative of the adhesive's bond strength with tissue.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically. Additionally, two items that are “coupled” may be unitary with each other. To illustrate, components may be coupled by virtue of physical proximity, being integral to a single structure, or being formed from the same piece of material. Coupling may also include mechanical, thermal, electrical, communicational (e.g., wired or wireless), or chemical coupling (such as a chemical bond) in some contexts.

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. As used herein, the term “approximately” may be substituted with “within 10 percent of” what is specified. Additionally, the term “substantially” may be substituted with “within [a percentage] of” what is specified, where the percentage includes 0.1, 1, or 5 percent; or may be understood to mean with a design, manufacture, or measurement tolerance. The phrase “and/or” means and or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), and “include” (and any form of include, such as “includes” and “including”). As a result, an apparatus that “comprises,” “has,” or “includes” one or more elements possesses those one or more elements, but is not limited to possessing only those one or more elements. Likewise, a method that “comprises,” “has,” or “includes” one or more steps possesses those one or more steps, but is not limited to possessing only those one or more steps.

Any aspect of any of the systems, methods, and article of manufacture can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, it will be understood that the term “wherein” may be used interchangeably with “where.”

Further, a device or system that is configured in a certain way is configured in at least that way, but it can also be configured in other ways than those specifically described. The feature or features of one embodiment may be applied to other embodiments, even though not described or illustrated, unless expressly prohibited by this disclosure or the nature of the embodiments.

Some details associated with the aspects of the present disclosure are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

A further understanding of the nature and advantages of the present disclosure may be realized by reference to the following drawings. The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 is a block diagram of an example of a therapy system for providing oxygenated hemoglobin;

FIG. 2 is a schematic diagram of an example of a therapy system for providing oxygenated hemoglobin;

FIG. 3 is a schematic diagram of an example of a mixing device for mixing oxygen and hemoglobin;

FIG. 4 is a schematic diagram of another example of a mixing device for mixing oxygen and hemoglobin;

FIG. 5 is a schematic diagram of another example of a mixing device for mixing oxygen and hemoglobin;

FIG. 6 is a diagram of example of oxygen and hemoglobin affinity curves;

FIG. 7A is a diagram of an example of a therapy system for providing oxygenated hemoglobin;

FIG. 7B is a cross section of a tube of the therapy system of FIG. 7A;

FIG. 8 is a block diagram of a control system for an oxygenated hemoglobin therapy system;

FIG. 9 is a block diagram of an example of a kit for oxygenated hemoglobin therapy systems;

FIG. 10 is a block diagram of an example of a kit for oxygenated hemoglobin therapy systems;

FIG. 11 is a flowchart illustrating an example of a method of providing oxygenated hemoglobin therapy; and

FIG. 12 is a flowchart illustrating another example of a method of providing oxygenated hemoglobin therapy.

### DETAILED DESCRIPTION

As used herein, the terms “tissue site” and “target tissue” as used herein can broadly refer to a wound (e.g., open or closed), a tissue disorder, and/or the like located on or within tissue, such as, for example, bone tissue, adipose tissue, muscle tissue, neural tissue, dermal tissue, vascular tissue, connective tissue, cartilage, tendons, ligaments, and/or the like. The terms “tissue site” and “target tissue” as used herein can also refer to a surrounding tissue area(s) and/or areas of tissue that are not necessarily wounded or exhibit a disorder, but include tissue that would benefit from tissue generation and/or tissue that may be harvested and transplanted to another tissue location. The terms “tissue site” and “target tissue” may also include incisions, such as a surgical incision. In some implementations, “target tissue” may correspond or refer to a wound, and “tissue site” may correspond or refer to a tissue area(s) surrounding and including the target tissue. Additionally, the term “wound” as used herein can refer to a chronic, subacute, acute, traumatic, and/or dehisced incision, laceration, puncture, avulsion, and/or the like, a partial-thickness and/or full thickness burn, an ulcer (e.g., diabetic, pressure, venous, and/or the like), flap, and/or graft. A wound may include chronic, acute, traumatic, subacute, and dehisced wounds, partial-thickness burns, ulcers (such as diabetic, pressure, or venous insufficiency ulcers), flaps, grafts, and fistulas, for example.

The term “positive-pressure” (or “hyperbaric”) as used herein generally refers to a pressure greater than a local ambient pressure, such as the ambient pressure in a local environment external to a sealed therapeutic environment (e.g., an internal volume). In most cases, this positive-pressure will be greater than the atmospheric pressure at which the patient is located. Alternatively, the positive-pressure may be greater than a hydrostatic pressure associated with tissue at the tissue site. Unless otherwise indicated, values of pressure stated herein are gauge pressures. References to increases in positive-pressure typically refer to an increase in absolute pressure, and decreases in positive-pressure typically refer to a decrease in absolute pressure. Additionally, the process of increasing pressure may be described illustratively herein as “applying”, “delivering,” “distributing,” “generating”, or “providing” positive-pressure, for example.

The term “reduced-pressure” (and “negative-pressure” or “hypobaric”) as used herein generally refers to a pressure less than a local ambient pressure, such as the ambient pressure in a local environment external to a sealed therapeutic environment (e.g., an internal volume). In most cases, this reduced-pressure will be less than the atmospheric pressure at which the patient is located. Alternatively, the reduced-pressure may be less than a hydrostatic pressure associated with tissue at the tissue site. Unless otherwise indicated, values of pressure stated herein are gauge pressures. References to increases in reduced-pressure typically refer to a decrease in absolute pressure, and decreases in reduced-pressure typically refer to an increase in absolute pressure. Additionally, the process of reducing pressure may be described illustratively herein as “applying”, “delivering,” “distributing,” “generating”, or “providing” reduced-pressure, for example.

The term “fluid” may refer to liquid, gas, air, or a combination thereof. The term “fluid seal,” or “seal,” means a seal adequate to maintain a pressure differential (e.g., positive-pressure or reduced-pressure) at a desired site given the particular pressure source or subsystem involved. Similarly, it may be convenient to describe certain features in terms of fluid “inlet” or “outlet” in such a frame of reference. However, the fluid path may also be reversed in some applications, such as by substituting a reduced-pressure source (negative or hypobaric pressure source) for a positive-pressure source, and this descriptive convention should not be construed as a limiting convention.

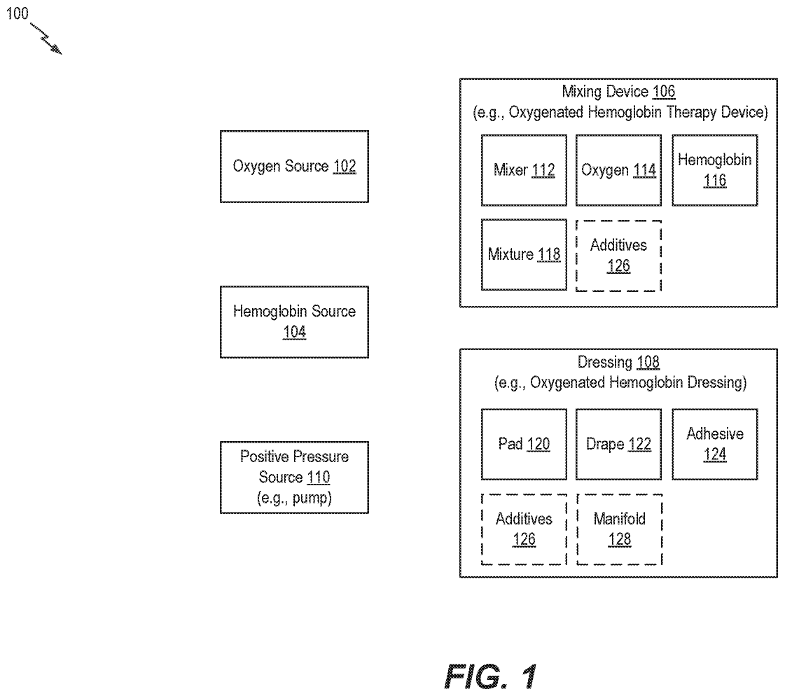

FIG. 1 illustrates a block diagram of an illustrative system 100 for providing oxygenated hemoglobin therapy. System 100 includes an oxygen source 102, a hemoglobin source 104, a mixing device 106, and a dressing 108. System 100 may optionally include a pressure source, such as positive pressure source 110 (e.g., a pump), a therapy device (e.g., 710), or both.

System 100 is configured to mix oxygen (02) and hemoglobin (Hb) to form an mixture, e.g., oxygenated hemoglobin, and to provide the mixture to dressing 108, such as a wound site thereof.

Oxygen source 102 may include or correspond to gaseous oxygen and a container therefor in some implementations. Additionally, or alternatively, oxygen source 102 may include or correspond to a pressurized oxygen container, an oxygen concentrator, or an oxygen collector, as illustrative, non-limiting examples

Hemoglobin source 104 may include or correspond to natural or synthesized hemoglobin (e.g., topical hemoglobin) and a container therefor. Topical hemoglobin includes hemoglobin that is configured to be directly applied to the body. In some implementations, the hemoglobin source 104 includes aerosolized hemoglobin, such as “hemoglobin spray.” In a particular implementation, the hemoglobin includes or correspond to carbonylated hemoglobin (CO-Hb). The hemoglobin (e.g., CO-Hb) may be oxygenated upon contact with and mixing with oxygen. The oxygenated hemoglobin increases healing and decreases recovery times.

Hemoglobin source 104 may also include or correspond to one or more types of hemoglobin, such as fetal hemoglobin (HbF), adult hemoglobin (HbA), or both. Fetal hemoglobin (HbF) has a higher affinity for oxygen and may promote increased efficacy.

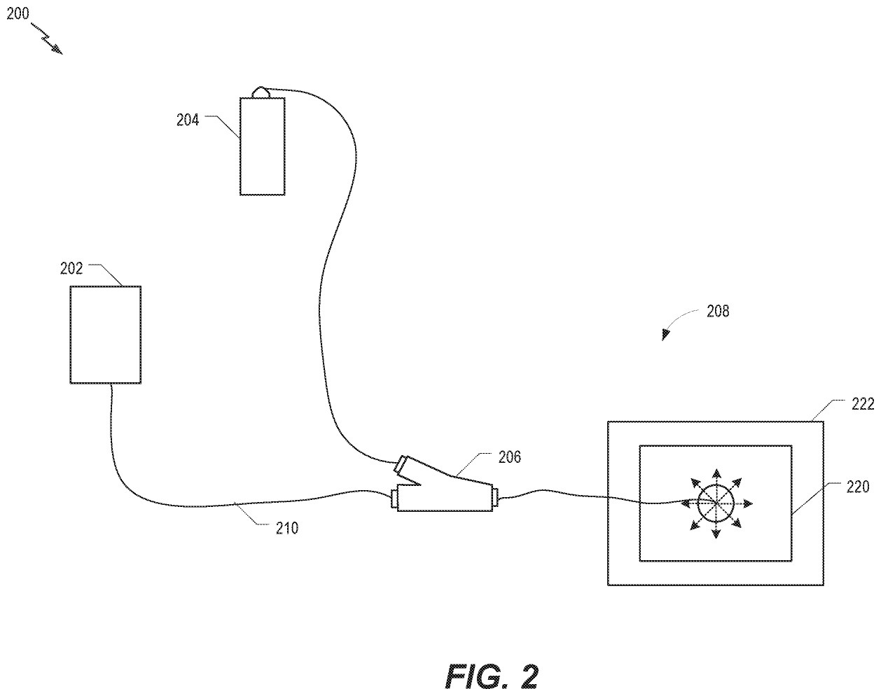

Mixing device 106 is coupled to oxygen source 102 and hemoglobin source 104 and includes a mixer 112. Mixing device 106 (e.g., mixer 112 thereof) may include or correspond to a device configured to mix the oxygen 114 and the hemoglobin 116, from their respective sources 102, 104, to form a mixture 118 of oxygen and hemoglobin, such as oxygenated hemoglobin. Examples of mixing devices 106 (e.g., mixer 112 thereof) are illustrated in FIGS. 2-5, and details of mixing device 106 are described further herein. Although mixing device 106 is illustrated separate from dressing 108 in FIG. 1, in other implementations mixing device 106 may be incorporated or integrated with dressing 108. Alternatively, mixing device 106 may be incorporated into a therapy device. In some implementations, mixing device 106 may include or receive an additive or additives 126 and mixes the additives 126 to form the mixture 118. The additives may include stabilizers or other ingredients to increase stability and/or efficacy of the hemoglobin or the mixture 118.

Dressing 108 may include or correspond to a wound dressing, such as pressurized wound dressing configured to provide pressurized wound therapy, such as positive-pressure therapy and/or oxygen therapy. As illustrated in the example of FIG. 1, dressing 108 includes a pad 120 (also referred to as a connector or a dressing connection pad), a drape 122, and an adhesive 124.

Pad 120 may be configured to couple dressing 108 to external devices, such as mixer 106 and/or therapeutic devices (e.g., a therapy device 710). Pad 120 may be selectively coupled to drape 122. To illustrate, pad 120 may be coupled to drape 122 via adhesive 124. In some implementations, pad 120 is directly coupled to drape 122 via adhesive 124, while in other implementations, one or more intervening layer are positioned between pad 120 and drape 122; in yet other implementations, a separate adhesive drape disposed over at least a portion of pad 120 and at least a portion of drape 122, as illustrative, non-limiting examples.

Drape 122 may be configured to couple dressing 108 to a tissue site and/or to provide a seal to create an enclosed space (e.g., an interior volume) corresponding to the tissue site. For example, drape 122 may be configured to provide a fluid seal between two components and/or two environments, such as between a sealed therapeutic environment and a local ambient environment. To illustrate, when coupled to the tissue site, drape 122 is configured to maintain a pressure differential at the tissue site. In some implementations, pressure differential may be provided by a therapy device, mixing device 106, pressure source 110, or another pressure source. Additionally, drape 122 may seal or block ambient light from transmission to the tissue site and/or adhesives attached thereto. Drape 122 may include a drape aperture that extends through drape 122 to enable fluid communication between mixing device 106, pad 120, and tissue site.

Drape 122 may be coupled to a tissue site via adhesives 124. To illustrate, drape 122 may be coupled to the pad 120 via a first adhesive and may be coupled to a tissue site via a second adhesive. Additionally, or alternatively, drape 122 may be coupled to a tissue site via a double-sided drape tape, paste, hydrocolloid, hydrogel, and/or other sealing device or element, as illustrative, non-limiting examples.

Adhesive 124 may include or correspond to a pressure sensitive adhesive. As an illustrative, non-limiting example, the adhesive 124 includes or correspond to switch adhesive, such as light switchable adhesive, a moisture switchable adhesive, a dual switchable adhesive, etc. A switchable adhesive may include one or more polymers and photo initiators, and/or polymerization initiators. The one or more polymers, the photo initiators, and the polymerization initiators may include or correspond to a polymer composition. Adhesive 124 may include a light curing system of a light switchable adhesive as described in International Patent Application Nos. PCT/US2018/049388 and PCT/US2018/060718, which are incorporated by reference herein to the extent they describe light switchable adhesives.

The one or more polymers may include chains of one or more monomers (e.g., polymer chains) and free monomers. The one or more polymers may include or correspond to an uncured or partially cured polymer composition and may be cured (or partially cured) responsive to receiving light from a light source (e.g., a light device) and/or moisture from a moisture source (e.g., a wipe). In some implementations, the one or more polymers are acrylic based, such as includes acrylate, urethane acrylate, alkoxy acrylate, and/or silicone acrylate based polymers and oligomers. The one or more polymers may include or further include polyether, polyurethane, methacrylate, or a combination thereof.

The photo initiators (e.g., first type curing initiators) are configured to cause cross-linking of monomers and/or polymer chains of the one or more polymers to increase a degree of cross linking of the one or more polymers or a degree of curing of the one or more polymers responsive to receiving light of a particular wavelength, i.e., first light. For example, the photo initiators are configured to generate free radicals (e.g., first free radicals) responsive to receiving first light from the light device. The free radicals are configured to active the one or more polymers (e.g., monomers or polymer chains thereof) to increase bonding between the one or more polymers, such as increase polymer chain lengths, reduce free monomers, or both.

As an illustrative, non-limiting example, the photo initiators include ultraviolet (UV) type photo initiators, i.e., photo initiators that are activated by light near or within the ultraviolet spectrum. Additionally, or alternatively, the photo initiators include visible light type photo initiators or infrared (IR) type photo initiators.

Similarly, the polymerization initiators (e.g., second type curing initiators) are configured to cause cross-linking of monomers and/or polymer chains of the one or more polymers to increase a degree of cross linking of the one or more polymers or a degree of curing of the one or more polymers responsive to receiving light of a particular wavelength. For example, the polymerization initiators are configured to increase curing responsive to receiving moisture from the moisture source. In some implementations, the polymerization initiators cause a condensation reaction responsive to receiving moisture from the moisture source. The polymerization initiators may proceed in a stepwise fashion to produce an addition reaction (generate an adduct) and release a byproduct. The addition reaction increases a chain length of the one or more polymers and may increase cross-linking of the one or more polymers. In other implementations, the polymerization initiators generate free radicals (e.g., second free radicals) responsive to receiving moisture from the moisture source. The free radicals are configured to active the one or more polymers (e.g., monomers or polymer chains thereof) to increase bonding between the one or more polymers, such as increase polymer chain lengths, reduce free monomers, or both.

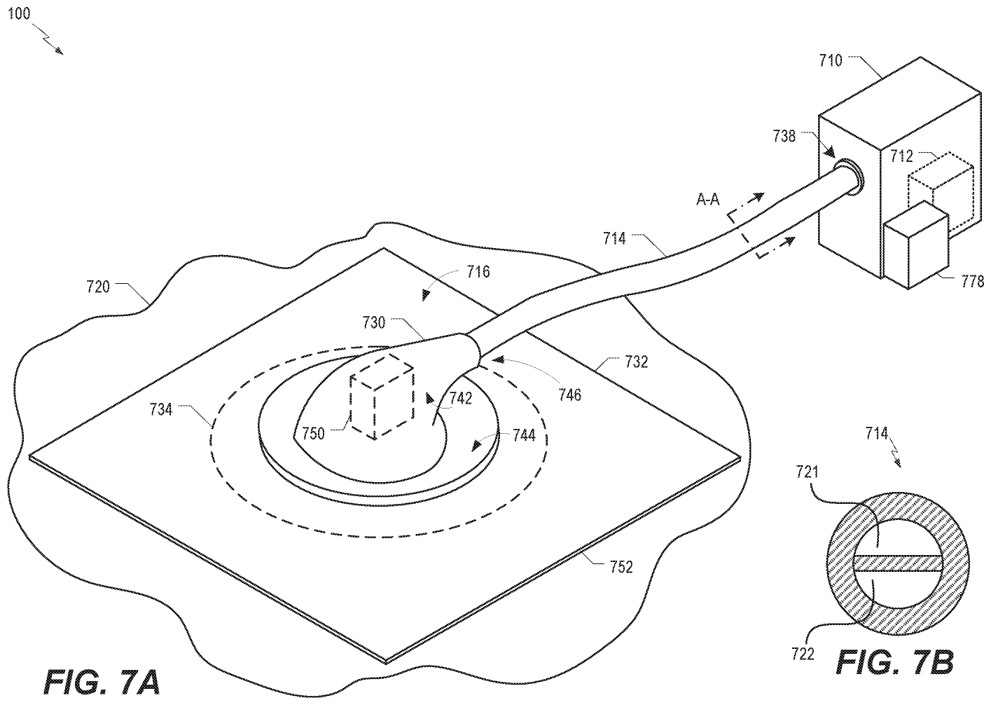

In some implementations, the adhesive 124 includes one or more additives 126. The additive 126 may include or correspond to additives to increase dissolution of the photo initiators, the polymerization initiators, or both in a particular polymer or to increase free radical production and/or curing. Dressing 108 optionally includes a manifold 128 and one or more other components. Examples of dressing 108, and components thereof, are described further with reference to FIG. 7A.

Pressure source 110 may include a negative-pressure source, such as a pump, or a positive-pressure source, (such as a pump, a pressurized oxygen container, an oxygen concentrator, or an oxygen collector) configured to be actuatable (and/or actuated) to apply a pressure differential relative to ambient conditions to dressing 108. As illustrative, non-limiting examples, positive-pressure applied to a tissue site may typically ranges between 5 millimeters mercury (mm Hg) (667 pascals (Pa)) and 30 mm Hg (4.00 kilo (k) Pa). Common therapeutic ranges are between 10 mm Hg (1.33 kPa) and 25 mm Hg (3.33 kPa). As illustrative, non-limiting examples, reduced-pressure applied to a tissue site may typically ranges between −5 millimeters mercury (mm Hg) (−667 pascals (Pa)) and −500 mm Hg (−66.7 kilo (k) Pa). Common therapeutic ranges are between −75 mm Hg (−9.9 kPa) and −300 mm Hg (−39.9 kPa).

Additionally, or alternatively, pressure source 110 may be configured to transport oxygen, hemoglobin, or both. In some such implementations, pressure source 110 may be configured to mix the oxygen and hemoglobin, and optionally other additives.

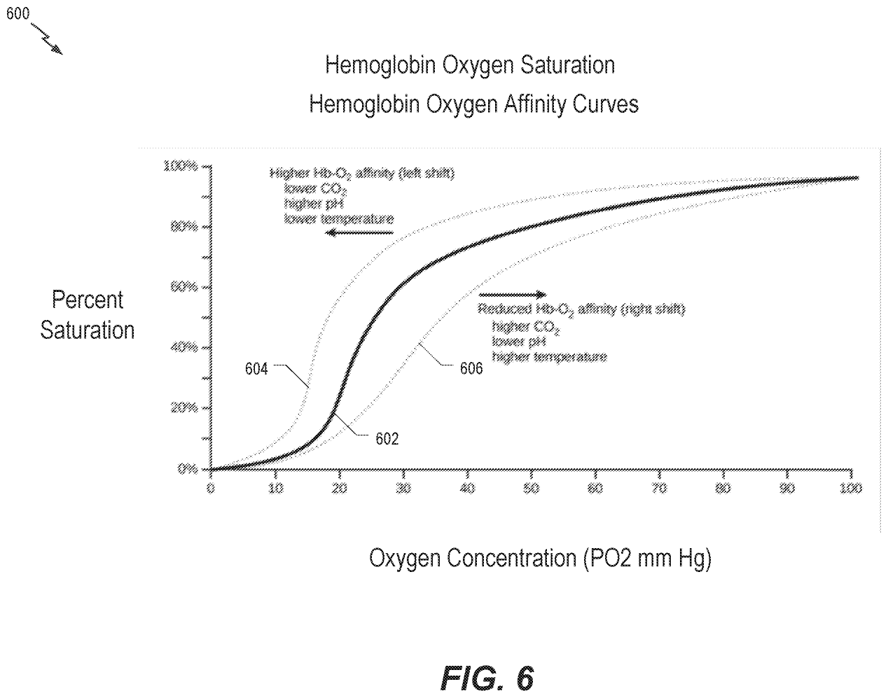

Prior to operation, components of system 100 may be coupled to each other, such as by tubing (such as tubing 210 and not shown in FIG. 1). Once coupled together, system 100 may provide therapy to a patient via dressing 108. During operation, i.e., while dressing 108 is attached to a tissue site of a patient, pressure source 110 provides or generates a pressure differential. The pressure differential causes oxygen from the oxygen source 102 and hemoglobin from the hemoglobin source 104 to enter mixing device 106. Mixing device 106 mixes the hemoglobin and the oxygen to form a mixture, such as oxygenated hemoglobin. The mixing device 106 provides the mixture to the dressing 108. The oxygenated hemoglobin may deliver oxygen to a wound of the patient. The oxygenated hemoglobin provides a higher transfer of oxygen to cells of the wound than oxygen alone, as described further with reference to FIG. 6.

In some implementations, after delivery of oxygen to the wound by the hemoglobin, the hemoglobin may return to the mixing device 106 via a return tubing or lumen to be re-oxygenated or may receive more oxygen within dressing 108 (e.g., near the wound) from oxygen source 102. Additionally, negative pressure therapy may be applied to dressing 108 to provide negative-pressure wound therapy, such as to remove exudate from the wound. To illustrate, positive and negative pressure may be cycled to increase healing and recovery. After therapy is complete, one or more components of the system 100 may be decoupled from one another or replaced.

Thus, system 100 describes a therapy system for providing oxygenated hemoglobin to a dressing. Oxygenated hemoglobin is suitable for use in medical devices, such as bandages, drapes, dressings, and wound closures. System 100 enables improved wound therapy efficacy, thereby decreasing recovery times, reducing complications, and reducing patient discomfort. Accordingly, system 100 may enable improved wound care and therapy, thereby advancing patient comfort and confidence in the treatment.

FIG. 2 illustrates a schematic diagram of an illustrative system 200 for providing oxygenated hemoglobin therapy. System 200 may include or correspond to system 100. System 200 includes an oxygen source 202, a hemoglobin source 204, a mixing device 206, a dressing 208, and tubing 210.

In the example of FIG. 2, oxygen source 202 includes an oxygen generator, such as a continuous oxygen diffusion device. Oxygen source 202 is configured to provide pure (e.g., medically pure, such as 90-100 percent pure) humidified oxygen to mixing device 206. The oxygen source 202 may include a pump or other pressure source and may be configured to provide pressurized oxygen to mixing device 206.

Hemoglobin source 204 includes or corresponds to aerosol hemoglobin or compressed hemoglobin in the example of FIG. 2. Hemoglobin source 204 is configured to provide hemoglobin or a hemoglobin mixture to mixing device 206. For example, the hemoglobin mixture may include 1 to 50 percent hemoglobin in a solution or carrier. The hemoglobin source 204 may store the hemoglobin or the hemoglobin mixture under pressure and may be configured to provide a pressurized hemoglobin mixture to mixing device 206.

Mixing device 206 is coupled to the oxygen source 202 and to the hemoglobin source 204 via tubing 210. As illustrated in FIG. 2, mixing device 206 includes two inlets and a single outlet. Mixing device 206 includes a first outlet corresponding to oxygen source 202 and a second outlet corresponding to hemoglobin source 204. To illustrate, a first tube of tubing 210 couples oxygen source 202 to the first inlet and a second tube of tubing 210 couples hemoglobin source 204 to the second inlet of mixing device 206. Mixing device 206 mixes the oxygen and the hemoglobin to form the mixture, and provides the mixture via the outlet to the dressing 208.

Dressing 208 includes a pad 220 and a drape 222, such as pad 120 and drape 122. As illustrated in the example of FIG. 2, pad 220 (e.g., a connector or connector pad) is coupled to the outlet of the mixing device 206 via a third tube of tubing 210. Pad 220 is positioned in an interior of dressing 208 and provides/distributes the mixture to the tissue site corresponding to dressing 208. As illustrated in the example of FIG. 2, the pad 220 is positioned in center of dressing 208 and the mixture moves radially away from the center of the dressing 208 to the tissue site. In other implementations, system 200 may optionally include a pressure source, a therapy device (e.g., 710), or both.

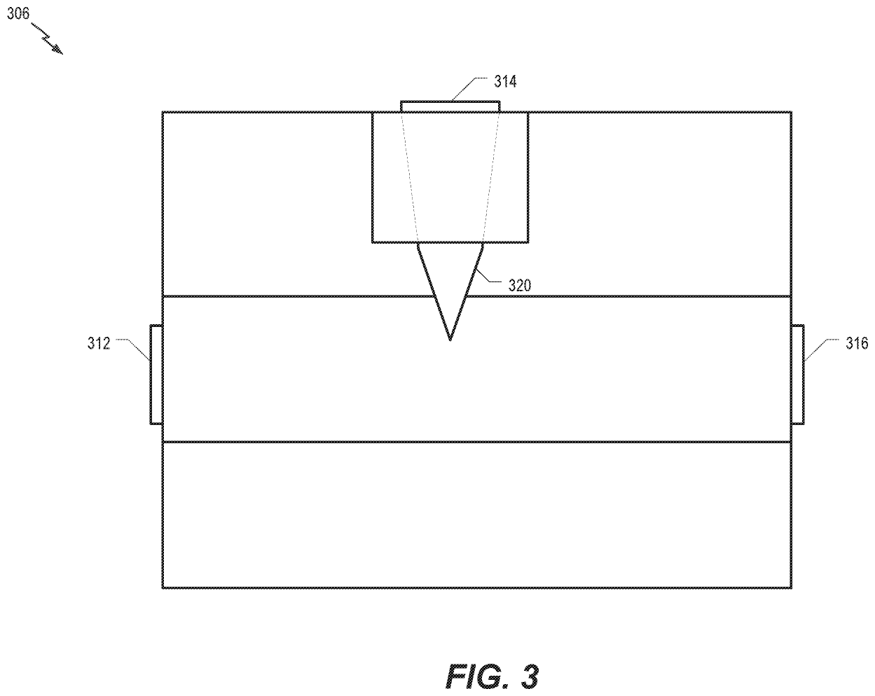

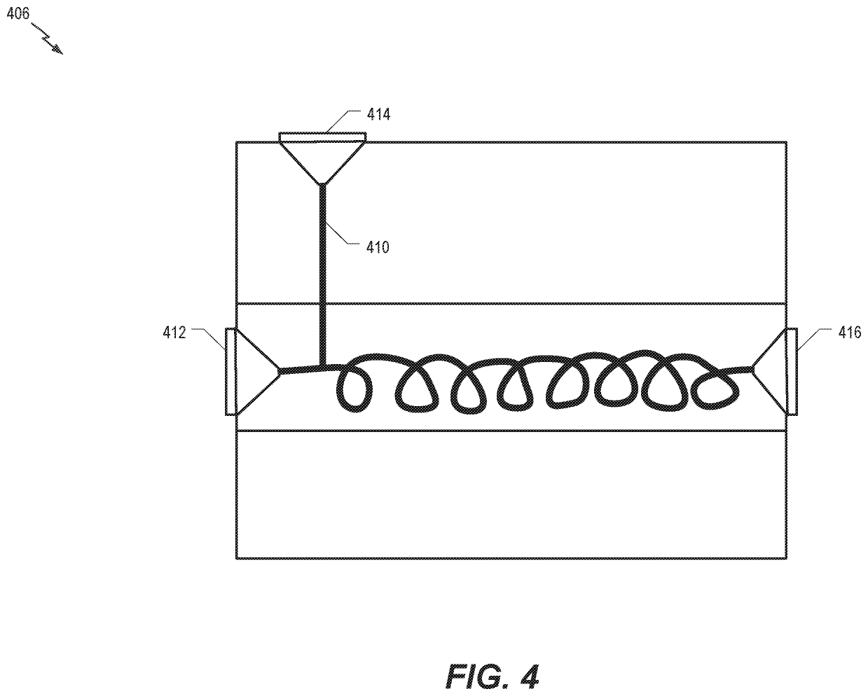

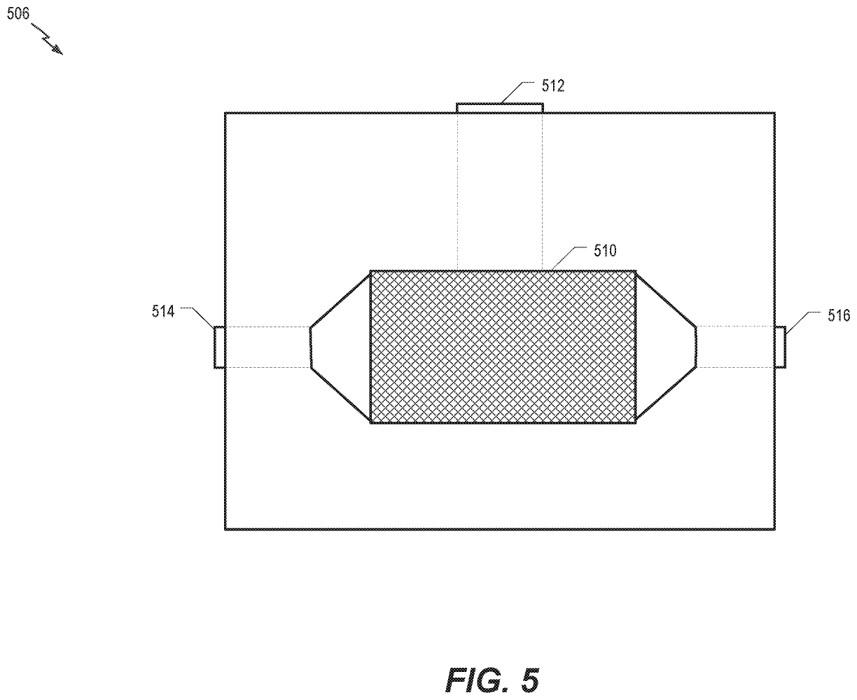

FIGS. 3-5 illustrate schematic diagrams of various examples of a mixing device for hemoglobin therapy, such as mixing device 106 or 206 (e.g., mixer 112 thereof). FIG. 3 illustrates an example of a spray type mixer, FIG. 4 illustrates an example of a spiral conveyor type mixer, and FIG. 5 illustrates an example of a porous exchange mixer.

Referring to FIG. 3, a diagram of a spray mixer 306 is illustrated. Spray mixer 306 includes an air input 312, a pressurized hemoglobin input 314, and an output 316. Air input 312 is arranged orthogonal to pressurized hemoglobin input 314. Such an arrangement (e.g., an intersecting or non-parallel arrangement) may increase turbulence and mass transfer within spray mixer 306, which promotes mixing of the oxygen and hemoglobin.

In the example of FIG. 3, pressurized hemoglobin input 314 includes a nozzle which increases a pressure of the hemoglobin as the hemoglobin travels from the hemoglobin source or inlet side to the dressing or exit side. Additionally, the increase in pressure of the hemoglobin by the pressurized hemoglobin input 314 increases mixing of the oxygen and the hemoglobin.

Referring to FIG. 4, a diagram of a spiral conveyor mixer 406 is illustrated. Spiral conveyor mixer 406 includes spiral tubing 410, an air input 412, a pressurized hemoglobin input 414, and an output 416.

In the example of FIG. 4, spiral tubing 410 includes a single tube or conduit formed into multiple spirals with two inlets and a single outlet. The non-linear path (e.g., spirals) of spiral tubing 410 may increase turbulent flow and mass transfer, which promotes mixing of the oxygen and hemoglobin.

Referring to FIG. 5, a diagram of a porous exchange mixer 506 is illustrated. Porous exchange mixer 506 includes a porous polymeric material 510, an air input 512, a pressurized hemoglobin input 514, and an output 516.

Porous polymeric material 510 may include or correspond to a polymer material, such as a polymer foam. The pores of the porous polymeric material 510, such as the uneven or random pores and pathways that connect the pores, may increase turbulent flow and mass transfer, which promotes mixing of the oxygen and hemoglobin. Additionally, in some implementations, the porous polymeric material 510 may act as a catalyst for the oxygenation of the hemoglobin.

As illustrated in the example of FIG. 5, the pressurized hemoglobin input 514 is arranged in a length or longitudinal direction of porous polymeric material 510, and the air input 512 is arranged orthogonal to the pressurized hemoglobin input 514 (i.e., downwards in FIG. 5).

Although specific example of mixers are illustrated in FIGS. 2-5, the mixers may include other types of mixers or combinations of mixers of FIGS. 2-5. Other mixers may include stirrers, agitators, pump mixers, fans, etc.

Referring to FIG. 6, an exemplary graph 600 illustrating Hemoglobin Oxygen affinity curves is shown. FIG. 6 illustrates a line graph illustrating percent saturation values of hemoglobin on a y-axis (vertical axis) and oxygen concentration values on an x-axis (horizontal axis) for hemoglobin oxygen saturation.

Solid line 602 depicts a base affinity curve for hemoglobin and oxygen. The dotted lines 604, 606, depict shifted affinity curves for hemoglobin and oxygen. Dotted line 604 depicts a left shifted affinity curve which indicate higher oxygen affinity; dotted line 606 depicts a right shifted affinity curve which indicates lower oxygen affinity. A higher oxygen affinity may be caused by lower carbon dioxide, higher pHs, and lower temperatures, while lower oxygen affinity may be caused by higher carbon dioxide, lower pHs, and higher temperatures.

The Hemoglobin Oxygen affinity curves shown in FIG. 6 illustrate that as a hemoglobin absorbs more oxygen, its affinity or ability to absorb more oxygen increases. As an illustrative example, a hemoglobin molecule may bind with four oxygen molecules. In such an example, the hemoglobin has a higher affinity for (e.g., to absorb) a fourth or last oxygen molecule when the hemoglobin molecule is already bound to (e.g., includes) three oxygen molecules, as compared to an affinity for a first oxygen molecule or a second oxygen molecule.

FIG. 7A shows a perspective view of an illustrative system 700 (e.g., a therapy system) for providing wound therapy. System 700 may include oxygenated hemoglobin, such as oxygen and hemoglobin for forming oxygenated hemoglobin, a therapy device 710, a canister 712, a tube 714, and a dressing 716. As an illustrative example, system 700 includes a mixer 750 as part of dressing 716 (e.g., connector 730 thereof). For example, a mixer 750 is attached to a body 742 of connector 730.

Alternatively, mixer 750 may be included as part of drape 732 of dressing 716. Drape 732 includes a protective film 792 removably coupled to the drape layer 794 opposite the adhesive, and the protective film 792 and drape layer 794 correspond to a compound film 752.

System 700 is configured to provide therapy (e.g., oxygen therapy, positive-pressure therapy, negative-pressure therapy, or a combination thereof) at a tissue site 720 associated with a target area of a patient. For example, dressing 716 may be in fluid communication with tissue site 720 and may be in fluid communication with therapy device 710 via tube 714. In some implementations, system 700 may include one or more components commercially available through and/or from KCI USA, Inc. of San Antonio, Tex., U.S.A., and/or its subsidiary and related companies (collectively, “KCI”).

Therapy device 710 (e.g., a treatment apparatus) is configured to provide therapy to tissue site 720 via tube 714 and dressing 716. For example, therapy device 710 may include a pressure source (e.g., a negative-pressure source, such as a pump, or a positive-pressure source, such as a pressurized oxygen container, an oxygen concentrator, or an oxygen collector) configured to be actuatable (and/or actuated) to apply pressure differential relative to ambient conditions to dressing 716. As illustrative, non-limiting examples, positive-pressure applied to a tissue site may typically ranges between 5 millimeters mercury (mm Hg) (667 pascals (Pa)) and 30 mm Hg (4.00 kilo (k) Pa). Common therapeutic ranges are between 10 mm Hg (1.33 kPa) and 25 mm Hg (3.33 kPa). As illustrative, non-limiting examples, reduced-pressure applied to a tissue site may typically ranges between −5 millimeters mercury (mm Hg) (−667 pascals (Pa)) and −500 mm Hg (−66.7 kilo (k) Pa). Common therapeutic ranges are between −75 mm Hg (−9.9 kPa) and −300 mm Hg (−39.9 kPa).

In some implementations, therapy device 710 may alternate between providing positive-pressure therapy and negative-pressure therapy to the dressing 716, may provide positive-pressure therapy to a first portion of the dressing 716 and negative-pressure therapy to a second portion of the dressing 716, may provide no positive or negative pressure, or a combination thereof. In some such implementations, the therapy device 710 can provide positive-pressure therapy and negative-pressure therapy to the dressing 716 at the same time (e.g., partially concurrently).

As illustrated in FIG. 7A, therapy device 710 includes canister 712 to provide hemoglobin to tissue site 720. Although canister 712 is illustrated as being internal to and/or integrated with therapy device 710, in other implementations, canister 712 is external to therapy device 710. Therapy device 710 also includes oxygen generation device 778. Oxygen generation device 778 is configured to generate oxygen and provide the oxygen to dressing 716 via tube 714, such as via a dedicated oxygen lumen thereof. Although oxygen generation device 778 is illustrated as being external to therapy device 710, in other implementations, oxygen generation device 778 internal to and/or integrated with therapy device 710. Therapy device 710 may include one or more additional canisters to receive fluid from tissue site 720 or to store oxygen or a mixture of oxygen and hemoglobin in other implementations.

Therapy device 710 may also include one or more other components, such as a sensor, a processing unit (e.g., a processor), an alarm indicator, a memory, a database, software, a display device, a user interface, a regulator, and/or another component, that further facilitate positive-pressure therapy. Additionally, or alternatively, therapy device 710 may be configured to receive fluid, exudate, and or the like via dressing 716 and tube 714. Therapy device 710 may include one or connectors, such as a representative connector 738. Connector 730 is configured to be coupled to tube 714. Additionally, or alternatively, therapy device 710 may include one or more sensors, such a pressure sensor (e.g., a pressure transducer). The one or more sensors may be configured to enable therapy device 710 to monitor and/or sense a pressure associated with tube 714 and/or dressing 716.

Tube 714 includes one or more lumens (e.g., one or more through conduits), such as a single lumen conduit or multiple single-lumen conduits. Tube 714 (e.g., a least one of the one or more lumens) is configured to enable fluid communication between therapy device 710 and dressing 716. For example, fluid(s) and/or exudate can be communicated between therapy device 710 and dressing 716, and/or one or more pressure differentials (e.g., positive-pressure, negative pressure, or both) can be applied by therapy device 710 to dressing 716. As an illustrative, non-limiting illustration, tube 714 is configured to deliver at least pressurized oxygen from therapy device 710 to dressing 716 to establish positive-pressure. Communication of fluid(s) and application of a pressure differential can occur separately and/or concurrently.

In some implementations, tube 714 may include multiple lumens, such as a primary lumen (e.g., a positive-pressure/fluid lumen) for application of positive-pressure and/or communication of fluid, and one or more secondary lumens proximate to or around the primary lumen. The one or more secondary lumens (e.g., one or more ancillary/peripheral lumens) may be coupled to one or more sensors (of therapy device 710), coupled to one or more valves, as an illustrative, non-limiting example. Although tube 714 is described as a single tube, in other implementations, system 700 may include multiple tubes, such as multiple distinct tubes coupled to therapy device 710, dressing 716, or both.

As used herein, a “tube” broadly refers to a tube, pipe, hose, conduit, or other structure with one or more lumens adapted to convey fluid, exudate, and/or the like, between two ends. In some implementations, a tube may be an elongated, cylindrical structure with some flexibility; however, a tube is not limited to such a structure. Accordingly, tube may be understood to include a multiple geometries and rigidity. Tube 714 includes one or more lumens (e.g., one or more through conduits), such as a single lumen conduit or multiple single-lumen conduits. Tube 714 (e.g., a least one of the one or more lumens) is configured to enable fluid communication between therapy device 710 and dressing 716. For example, fluid(s) and/or exudate can be communicated between therapy device 710 and dressing 716, and/or one or more pressure differentials (e.g., positive-pressure, negative pressure, or both) can be applied by therapy device 710 to dressing 716. As an illustrative, non-limiting illustration, tube 714 is configured to deliver at least pressurized oxygen from therapy device 710 to dressing 716 to establish positive-pressure. Communication of fluid(s) and application of a pressure differential can occur separately and/or concurrently.

Referring to FIG. 7B, an illustrative example of a cross-section of tube 714 (in which tube 714 comprises two lumens) along line A-A of FIG. 7A is shown. Tube 714 may include a primary lumen 721 (e.g., a positive-pressure/fluid lumen) and a secondary lumen 722. In a particular example, primary lumen 721 is configured to provide oxygen and secondary lumen 733 is configured to provide hemoglobin to dressing 716. In other implementations, tube 714 may include one or more other secondary lumens, such as a negative-pressure/fluid lumen, a return lumen, one or more sense lumens, etc., or a combination thereof. Although tube 714 has been described and/or shown as having a circular cross-sectional shape, in other implementations, tube 714 may have a cross-sectional shape other than a circle, such as an oval, triangle, quadrilateral, pentagon, star, or another shape, as illustrative, non-limiting examples.

Referring to FIG. 7A, dressing 716 includes a connector 730 (also referred to as a dressing connection pad or a pad), a drape 732, and a manifold 734 (also referred to as a distribution manifold or an insert). Drape 732 may be coupled to connector 730. To illustrate, drape 732 may be coupled to connector 730 via an adhesive, a separate adhesive drape over at least a portion of connector 730 and at least a portion of drape 732, or a combination thereof, as illustrative, non-limiting examples.

Drape 732 may be configured to couple dressing 716 at tissue site 720 and/or to provide a seal to create an enclosed space (e.g., an interior volume) corresponding to tissue site 720. For example, drape 732 may be configured to provide a fluid seal between two components and/or two environments, such as between a sealed therapeutic environment and a local ambient environment. To illustrate, when coupled to tissue site 720, drape 732 is configured to maintain a pressure differential (provided by a positive-pressure source or a negative-pressure source) at tissue site 720. Drape 732 may include a drape aperture that extends through drape 732 to enable fluid communication between device and target tissue. Drape 732 may be configured to be coupled to tissue site 720 via an adhesive, such as a medically acceptable, pressure-sensitive adhesive that extends about a periphery, a portion, or an entirety of drape 732. Additionally, or alternatively, drape 732 may be coupled to tissue site 720 via a double-sided drape tape, paste, hydrocolloid, hydrogel, and/or other sealing device or element, as illustrative, non-limiting examples.

Drape 732 may include an impermeable or semi-permeable, elastomeric material, as an illustrative, non-limiting example. In some implementations, drape 732 may be liquid/gas (e.g., moisture/vapor) impermeable or semi-permeable. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones. In some implementations, drape 732 may include the “V.A.C.® Drape” commercially available from KCI. Additional, specific non-limiting examples of materials of drape 732 may include a silicone drape, 3M Tegaderm® drape, and a polyurethane (PU) drape such as one available from Avery Dennison Corporation of Pasadena, Calif. An additional, specific non-limiting example of a material of the drape 732 may include a 30 micrometers (μm) matt polyurethane film such as the Inspire™ 2317 manufactured by Exopack™ Advanced Coatings of Matthews, N.C.

Manifold 734 is configured to be positioned on and/or near tissue site 720, and may be secured at the tissue site 720, such as secured by drape 732. The term “manifold” as used herein generally refers to a substance or structure that may be provided to assist in applying a pressure differential (e.g., positive-pressure differential) to, delivering fluids to, or removing fluids and/or exudate from a tissue site and/or target tissue. The manifold typically includes a plurality of flow channels or pathways that distribute fluids provided to and removed from the tissue site. In an illustrative implementation, the flow channels or pathways are interconnected to improve distribution of fluids provided to or removed from the tissue site. Manifold 734 may be a biocompatible material that may be capable of being placed in contact with the tissue site and distributing positive and/or negative-pressure to the tissue site. Manifold 734 may include, without limitation, devices that have structural elements arranged to form flow channels, such as foam, cellular foam, open-cell foam, porous tissue collections, liquids, gels, and/or a foam that includes, or cures to include, flow channels, as illustrative, non-limiting examples. Additionally, or alternatively, manifold may include polyethylene, a polyolefin, a polyether, polyurethane, a co-polyester, a copolymer thereof, a combination thereof, or a blend thereof.

In some implementations, manifold 734 is porous and may be made from foam, gauze, felted mat, or other material suited to a particular biological application. In a particular implementation, manifold 734 may be a porous foam and may include a plurality of interconnected cells or pores that act as flow channels. The foam (e.g., foam material) may be either hydrophobic or hydrophilic. As an illustrative, non-limiting example, the porous foam may be a polyurethane, open-cell, reticulated foam such as GranuFoam® material manufactured by Kinetic Concepts, Incorporated of San Antonio, Tex.

In some implementations, manifold 734 is also used to distribute fluids such as medications, antibacterials, growth factors, and other solutions to the tissue site. Other layers may be included in or on manifold 734, such as absorptive materials, wicking materials, hydrophobic materials, and hydrophilic materials. In an implementation in which the manifold 734 includes a hydrophilic material, manifold 734 may be configured to wick fluid away from tissue site 720 and to distribute positive-pressure to tissue site 720. The wicking properties of manifold 734 may draw fluid away from the tissue site 720 by capillary flow or other wicking mechanisms. An illustrative, non-limiting example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether and/or foams that have been treated or coated to provide hydrophilicity.

In some implementations, manifold 734 is constructed from bioresorbable materials that do not have to be removed from tissue site 720 following use of the system 700. Suitable bioresorbable materials may include, without limitation, a polymeric blend of polylactic acid (PLA) and polyglycolic acid (PGA). The polymeric blend may also include without limitation polycarbonates, polyfumarates, and capralactones. Manifold 734 may further serve as a scaffold for new cell-growth, or a scaffold material may be used in conjunction with manifold 734 to promote cell-growth. A scaffold may be a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials. Although a manifold 734 is illustrated in FIG. 7A, in other implementations, dressing 716 does not include manifold 734. In such implementations, drape 732 of dressing 716 is coupled to connector 730.

Connector 730 includes a body 742 (e.g., a housing) and a base 744, and is configured to be coupled to tube 714 via an interface 746 (e.g., a port). Base 744 is configured to be coupled to dressing 716. For example, base 744 may be coupled, such as via an adhesive, to drape 732 and/or manifold 734. In some implementations, base 744 comprises a flange that is coupled to an end of body 742 and/or is integrally formed with body 742. Connector 730, such as body 742, base 744, interface 746, or a combination thereof, may be made of rigid material and/or a semi-rigid material. In a non-limiting example, connector 730 may be made from a plasticized polyvinyl chloride (PVC), polyurethane, cyclic olefin copolymer elastomer, thermoplastic elastomer, poly acrylic, silicone polymer, or polyether block amide copolymer. In some implementations, connector 730 is formed of a semi-rigid material that is configured to expand when under a force, such as positive-pressure greater than or equal to a particular amount of pressure. Additionally or alternatively, connector 730 may be formed of a semi-rigid material that is configured to collapse when under a force, such as reduced-pressure less than or equal to a threshold pressure.

Body 742 includes one or more channels or one or more conduits that extend from and/or are coupled to interface 746. To illustrate, body 742 may include a primary channel configured to be coupled in fluid communication with a primary lumen (e.g., 721) of tube 714. The primary channel may be coupled to a cavity (e.g., a tissue cavity partially defined by body 742) having an aperture open towards manifold 734 (and/or towards tissue site 720). For example, the primary channel may include a first opening associated with interface 746 and a second opening (distinct from the aperture of the cavity) associated with the cavity. Thus, the primary channel may define a through channel of body 742 to enable fluid communication between interface 746 and tissue site 720.

Body 742 includes a channel (e.g., a through channel) having a first aperture open opposite dressing 716 and a second aperture open towards dressing 716. For example, the first aperture is located on an outer surface side (e.g., an ambient environment surface) of connector 730 and the second aperture is located on an inner surface side (e.g., a tissue facing side) of connector 730. The second aperture is configured to be coupled to one or more lumens of tube 714, such as coupled via the cavity. Illustrative, non-limiting examples of commercially available connectors include a “V.A.C. T.R.A.C.® Pad,” or “Sensa T.R.A.C.® Pad” available from Kinetic Concepts, Inc. (KCI) of San Antonio, Tex.

During operation of system 700, dressing 716 is coupled to tissue site 720 over a wound. Additionally, dressing 716 is coupled to device 710 via tube 714. In some implementations, prior to coupling the dressing 716 to the tissue site 720, a bandage or a wound closure device is coupled to tissue site 720 proximate to a wound. The dressing 716 is then coupled over the bandage or wound closure device. The dressing 716 is coupled to tissue site 720 site via compound film 752. To illustrate, adhesive of the compound film 752 bonds the dressing 716 to the tissue site 720 responsive to pressure. In a particular implementation when the compound film 752 is included in or corresponds to drape 732, and the compound film 752 may seal a portion of tissue site 720, such as an interior volume of dressing 716.

A pressure differential, such as positive-pressure, can be generated and/or applied to dressing 716 (e.g., the interior volume of dressing 716) by a pressure source associated with device 710. When positive-pressure is generated and/or applied to dressing 716, oxygen and hemoglobin from device 710, such as from oxygen generation device 778 and canister 712, may be transported to dressing 716. The mixer 750 of the dressing 716 may receive the oxygen and hemoglobin from separate lumens 721, 722 of tube 714 and mix the oxygen and hemoglobin to form a mixture of oxygen and hemoglobin which transports/transfers the oxygen to the wound (e.g., cells thereof).

Furthermore, in some implementations, reduced-pressure can be applied to dressing 716 (e.g., the interior volume of dressing 716 or a second interior volume of the dressing 716) by a reduced-pressure source associated with device 710. When reduced-pressure is applied to dressing 716 (e.g., when vacuum pressure is generated, fluid, exudate, or other material within dressing 716 may be transported to canister 712 of device 710. After operation, such as completion of therapy, system 700 may be disconnected and components thereof removed from tissue site 720.

Thus, dressing 716 can include a mixer 750 to mix the oxygenated hemoglobin closer to the wound or tissue site and a number of components may be reduced. Such a system may be easier to setup and operate and may provide enhanced efficacy by mixing the oxygen and hemoglobin closer to the wound.

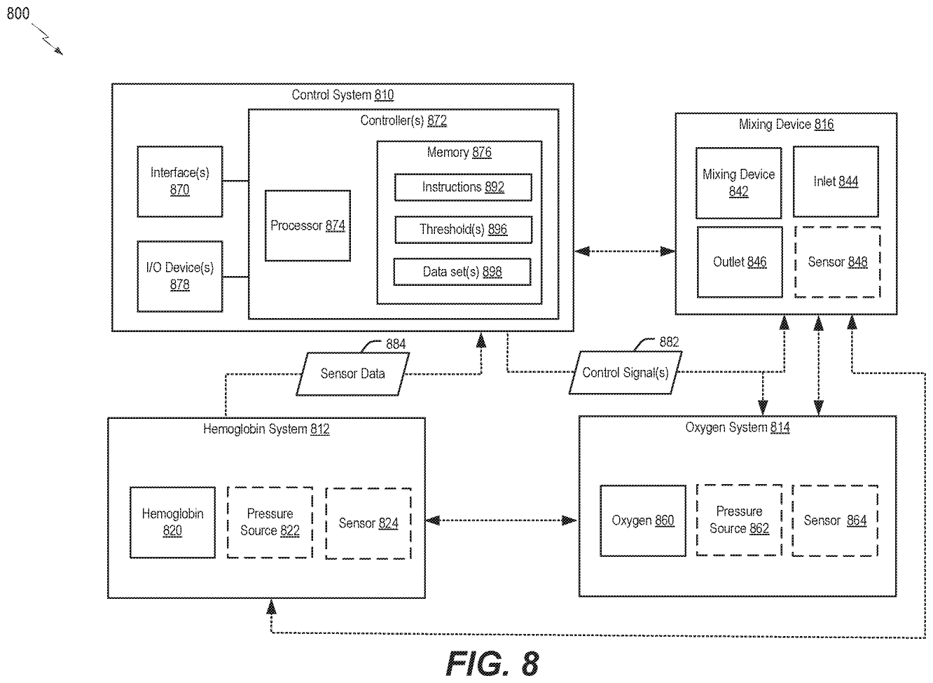

Referring to FIG. 8, a block diagram of a control system, system 800, for providing oxygenated hemoglobin to a dressing. In the example illustrated in FIG. 8, system 800 includes a control system 810, a hemoglobin system 812, an oxygen system 814, and mixing system 816. Control system 810 is configured to control one or more of systems 812-816, as described further herein.

Hemoglobin system 812 is configured to provide hemoglobin 820 to mixing system 816. For example, hemoglobin may be stored under pressure and in aerosol form, and hemoglobin system 812 is configured to control release and/or application of hemoglobin to mixing system 816. To illustrate, hemoglobin system 812 may include a pressurized canister of hemoglobin. A pressure source 822 may be configured to provide or transport the hemoglobin 820.

Oxygen system 814 is configured to apply oxygen 860 to mixing system 816. For example, Oxygen system 814 may generate oxygen or may release stored and pressurized oxygen to provide oxygen to mixing system 816. To illustrate, oxygen system 814 may include an oxygen collector or diffuser or an pressurized canister of oxygen. A pressure source 862 may be configured to provide or transport the oxygen 860. Optionally, one or more of systems 812, 814 include a sensor, such as sensor 824 or sensor 864.

Mixing system 816 includes a mixing device 842, at least one inlet 844, and an outlet 846. Mixing system 816 optionally includes a sensor 848. Mixing system 816 is configured to receive oxygen and hemoglobin and to mix the oxygen and the hemoglobin to form a mixture of oxygen and hemoglobin, such as oxygenated hemoglobin. Although listed as separate systems, systems 812-816 may be incorporated into a single system. For example, hemoglobin system 812 and oxygen system 814 may be incorporated into a single system. Additionally, system 800 may include one or more other systems, such as a pump system, a tubing system, a light switchable adhesive system, a drape formation system, a sterilization system, or a combination thereof.

Control system 810 includes one or more interfaces 870, one or more controllers, such as a representative controller 872, and one or more input/output (I/O) devices 878. Interfaces 870 may include a network interface and/or a device interface configured to be communicatively coupled to one or more other devices, such as hemoglobin system 812 or oxygen system 814. For example, interfaces 870 may include a transmitter, a receiver, or a combination thereof (e.g., a transceiver), and may enable wired communication, wireless communication, or a combination thereof. Although control system 810 is described as a single electronic device, in other implementations system 800 includes multiple electronic devices. In such implementations, such as a distributed control system, the multiple electronic devices each control a sub-system of system 800, such as hemoglobin system 812, oxygen system 814, or mixing system 816.

The one or more controllers (e.g., controller 872) includes one or more processors and one or more memories, such as representative processor 874 and memory 876. The one or more controllers may include or correspond to a hemoglobin controller, an oxygen controller, a mixing controller, or a combination thereof. For example, hemoglobin controller (e.g., processor 874) may be configured to generate and/or communicate one or more control signals 882 to hemoglobin system 812. Hemoglobin controller may be configured to control (or regulate) hemoglobin and/or an environment, such as an air quality, temperature, and/or pressure, within hemoglobin system 812 (e.g., an extruder thereof) and/or delivery/injection of hemoglobin from hemoglobin system 812. For example, hemoglobin controller may be configured to generate and/or communicate one or more control signals 882, such as environment control signals, ingredient delivery control signals, or a combination thereof, to hemoglobin system 812.

Oxygen controller may be configured to control (or regulate) oxygen and/or an environment, such as a temperature (e.g., heat) and/or pressure within oxygen system 814 (e.g., an applicator thereof) and/or delivery/injection of oxygen from oxygen system. For example, oxygen controller may be configured to generate and/or communicate one or more control signals 882, such as environment control signals, ingredient delivery control signals, or a combination thereof, to oxygen system 814.

Mixing controller may be configured to control (or regulate) mixing of oxygen and hemoglobin and/or an environment, such as a temperature (e.g., heat) and/or pressure within mixing system 814. For example, mixing controller may be configured to generate and/or communicate one or more control signals 882, such as environment control signals, ingredient delivery control signals, or a combination thereof, to mixing system 816.

Memory 876, such as a non-transitory computer-readable storage medium, may include volatile memory devices (e.g., random access memory (RAM) devices), nonvolatile memory devices (e.g., read-only memory (ROM) devices, programmable read-only memory, and flash memory), or both. Memory 876 may be configured to store instructions 892, one or more thresholds 896, and one or more data sets 898. Instructions 892 (e.g., control logic) may be configured to, when executed by the one or more processors 874, cause the processor(s) 874 to perform operations as described further here. For example, the one or more processors 874 may perform operations as described with reference to FIGS. 1-5, 7A, and 7B. The one or more thresholds 896 and one or more data sets 898 may be configured to cause the processor(s) 874 to generate control signals. For example, the processors 874 may generate and send control signals responsive to receiving sensor data from one or more of systems 812-816, such as exemplary sensor data 884 from oxygen system 814. The temperature or ingredient flow rate can be adjusted based on comparing sensor data to one or more thresholds 896, one or more data sets 898, or a combination thereof.

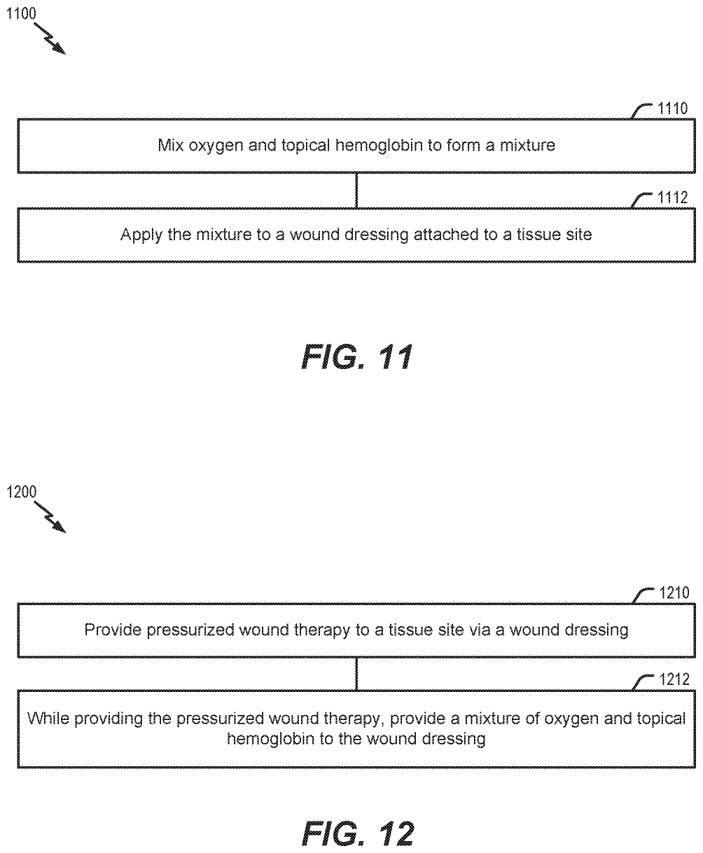

In some implementations, processor 874 may include or correspond to a microcontroller/microprocessor, a central processing unit (CPU), a field-programmable gate array (FPGA) device, an application-specific integrated circuits (ASIC), another hardware device, a firmware device, or any combination thereof. Processor 874 may be configured to execute instructions 892 to initiate or perform one or more operations described with reference to FIG. 1, FIG. 2, or FIG. 7, and/or one more operations of the methods of FIG. 11 or 12.

The one or more I/O devices 878 may include a mouse, a keyboard, a display device, the camera, other I/O devices, or a combination thereof. In some implementations, the processor(s) 874 generate and send control signals responsive to receiving one or more user inputs via the one or more I/O devices 878.

Control system 810 may include or correspond to an electronic device such as a communications device, a mobile phone, a cellular phone, a satellite phone, a computer, a tablet, a portable computer, a display device, a media player, or a desktop computer. Additionally, or alternatively, the control system 810 may include a personal digital assistant (PDA), a monitor, a computer monitor, a television, any other device that includes a processor or that stores or retrieves data or computer instructions, or a combination thereof.

During operation of system 800, hemoglobin system 812 releases stored hemoglobin to provide hemoglobin 820 to mixing system 816. To illustrate, controller 872 may send one or more control signals 882 to hemoglobin system 812. The control signals 882 may include signals configured to cause hemoglobin system 812 to release or adjust release of hemoglobin 820. To illustrate, control system 810 may send one or more signals 882 (e.g., environment control signals) to hemoglobin system 812 to adjust conditions (e.g., heat, pressure, air quality) of the hemoglobin system 812 or conditions (e.g., viscosity, temperature, etc.) of the hemoglobin 820.

Oxygen system 814 releases stored oxygen to provide oxygen 860 to mixing system 816. To illustrate, controller 872 may send one or more control signals 882 to oxygen system 814. The control signals 882 may include signals configured to cause oxygen system 814 to release or adjust release of oxygen 860. To illustrate, control system 810 may send one or more signals 882 (e.g., environment control signals) to oxygen system 814 to adjust conditions (e.g., heat, pressure, air quality) of the oxygen system 814 or conditions (e.g., viscosity, temperature, etc.) of the oxygen 860. Additionally, controller 872 may send one or more control signals 882 to oxygen system 814 to control generation of oxygen 860. For example, when oxygen system 814 includes or corresponds to an oxygen collector or source, controller 872 may send control signals to activate, stop, and/or adjust oxygen generation/collection.

After hemoglobin and oxygen are provided to mixing system 816, mixing system 816 mixes the hemoglobin and the oxygen to form oxygenated hemoglobin and the oxygenated hemoglobin (or components thereof) may be further processed by mixing system 816 before delivery to dressing, such as by filtering. To illustrate, controller 872 may send one or more control signals 882 to mixing system 816 to control mixing of oxygen and hemoglobin, delivery of the mixture to a dressing or a container for storage, or a combination thereof.

Thus, system 800 of FIG. 8 provides a control system for controlling generation of oxygenated hemoglobin and controlling oxygenated hemoglobin therapy. Accordingly, the present disclosure enables formation of oxygenated hemoglobin during therapy and providing oxygenated hemoglobin during positive pressure and/or oxygen therapy.

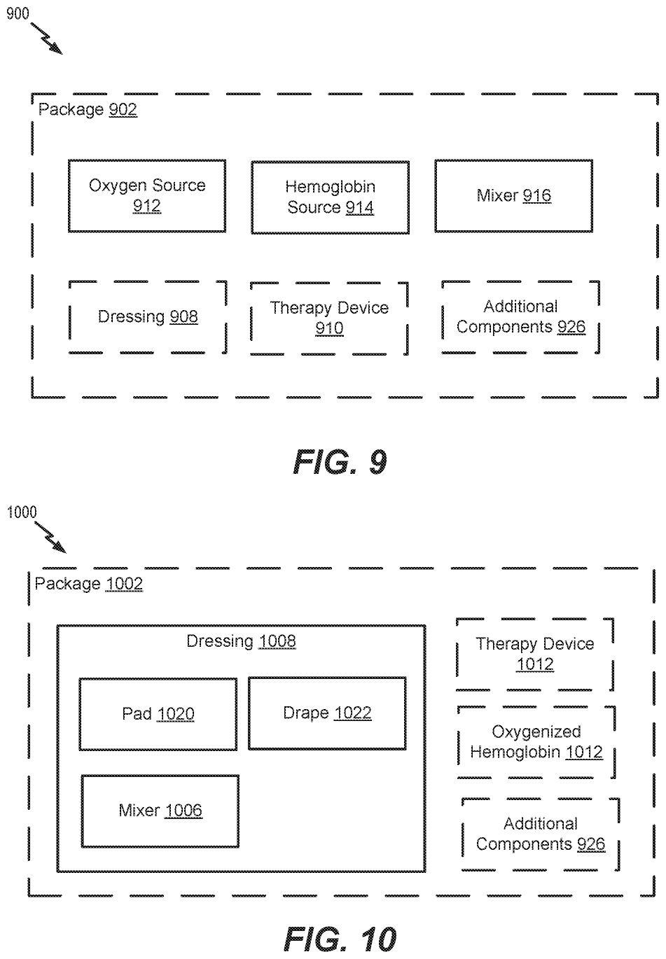

Referring to FIG. 9, a kit 900 for medical devices, such as a component of system 100, is illustrated. Kit 900 includes one or more of an oxygen source 912, a hemoglobin source 914, or a mixer 916. The oxygen source 912 may include or correspond to oxygen source 102 or oxygen source 202. The hemoglobin source 914 may include or correspond to hemoglobin source 104 or hemoglobin source 204. Alternatively, the oxygen and hemoglobin sources may be integrated as in FIG. 10, i.e., an oxygenated hemoglobin source. The mixer 916 may include or correspond to a mixer, mixer device, or mixer system as described herein, such as 106, 206, 206, 406, 506, 750, or 816.

In some implementations, kit 900 may further include a dressing 908, a therapy device 910, one or more components 926, or a combination thereof. Dressing 908 may include or correspond to dressing 108 or dressing 716. Therapy device 910 may include or correspond to therapy device 710. The one or more additional components 926 may include or correspond to adhesive, LSA, an LSA applicator, a light device for activating the LSA, gloves, antiseptic, medical adhesive, and/or other components.

In some implementations, kit 1000 may include a package 1002. For example, package 1002 may include a box, a bag, a container, or the like. Package 1002 may include the pad 1020 and/or the drape 1022, in addition to the one or more optional components described above. In some implementations, package 1002 may include a packaging medium (e.g., packaging material), such as foam, paper, or the like. Thus, FIG. 10 describes kit 1000 for a therapy device including a two-piece dressing with LSA.

Referring to FIG. 10, a kit 1000 for therapy devices, such as a component of system 100, is illustrated. Kit 1000 includes one or more wound dressings, or optionally components for forming one or more wound dressings, such as a pad 1020, a drape 1022, and/or a mixer 1006. The pad 1020 may include or correspond to pad 120 or connector 730. The drape 1022 may include or correspond to drape 122 or drape 732. In some implementations, the pad 1020 and the drape 1022 are separate, and in other implementations, the pad 1020 and the drape 1022 are coupled together by an adhesive, such as LSA, i.e., as in a pre-packaged dressing. When the pad 1020 and the drape 1022, the LSA may be provided separately, such as in a tub of adhesive (e.g., LSA).

In some implementations, kit 1000 includes oxygenated hemoglobin 1012, as illustrated. In other implementations, kit 1000 includes an oxygen source, a hemoglobin source, or both, as illustrated in FIG. 9.

In some implementations, kit 1000 further includes therapy device 1010, one or more components 1026, or a combination thereof. Therapy device 1010 may include or correspond to therapy device 710. The one or more additional components 1026 may include or correspond to adhesive, LSA, an LSA applicator, a light device for activating the LSA, gloves, antiseptic, medical adhesive, and/or other components.

In some implementations, kit 1000 may include a package 1002. For example, package 1002 may include a box, a bag, a container, or the like. Package 1002 may include the pad 1020 and/or the drape 1022, in addition to the one or more optional components described above. In some implementations, package 1002 may include a packaging medium (e.g., packaging material), such as foam, paper, or the like. Thus, FIG. 10 describes kit 1000 for a therapy device including a two-piece dressing with LSA.

FIG. 11 illustrates a method 1100 of providing oxygenated hemoglobin to a tissue site. The method 1100 may be performed by a patient or care provider using one or more components of system 100, system 700, or system 800 or by the one or more components of system 100 or system 700. Method 1100 includes mixing oxygen and topical hemoglobin to form a mixture, at 1210. For example, the oxygen may include or correspond to oxygen of oxygen source 102, and the topical hemoglobin may include or correspond to hemoglobin of hemoglobin source 104. The tissue site may include or correspond to tissue site 720. To illustrate, a mixing device 106 mixes the oxygen and the topical hemoglobin from sources 102, 104.

Method 1100 further includes applying the mixture to a wound dressing attached to a tissue site, at 1112. For example, the wound dressing may include or correspond to dressing 108. To illustrate, a mixing device 106 provides the oxygenated hemoglobin mixture to the dressing 108 via tubing, such as tubing 210.

Thus, method 1100 describes a method of providing oxygenated hemoglobin to a tissue site. The oxygenated hemoglobin enables improved oxygen delivery and wound therapy compared to oxygen or positive pressure therapy systems. Accordingly, the oxygenated hemoglobin therapy systems described herein may enable improved wound care and therapy, thereby advancing patient comfort and confidence in the treatment.

FIG. 12 illustrates a method 1200 of providing oxygenated hemoglobin to a tissue site. The method 1200 may be performed at or by one or more components of system 100, system 700, or system 800, such as a mixing device thereof. Method 1200 includes providing pressurized wound therapy to a tissue site via a wound dressing, at 1210. For example, the tissue site may include or correspond to tissue site 720, and the wound dressing may include or correspond to dressing 108. To illustrate, a mixing device 106 or container of oxygenated hemoglobin provides the oxygenated hemoglobin to the dressing 108.

Method 1200 further includes, while providing the pressurized wound therapy, providing a mixture of oxygen and topical hemoglobin to the wound dressing, at 1212. For example, the oxygen may include or correspond to oxygen of oxygen source 102, and the topical hemoglobin may include or correspond to hemoglobin of hemoglobin source 104. The mixture may include or correspond to oxygenated hemoglobin, as described herein. To illustrate, a mixing device 106 or a container of oxygenated hemoglobin provides the oxygenated hemoglobin to the dressing 108 while the dressing is attached to a tissue site of a patient and is providing therapy.

Thus, method 1200 describes another method of providing oxygenated hemoglobin to a tissue site. The oxygenated hemoglobin enables improved oxygen delivery and wound therapy compared to oxygen or positive pressure therapy systems. Accordingly, the oxygenated hemoglobin therapy systems described herein may enable improved wound care and therapy, thereby advancing patient comfort and confidence in the treatment.

It is noted that one or more operations described with reference to one of the methods of FIGS. 11 and 12 may be combined with one or more operations of another of FIGS. 11 and 12. For example, one or more operations of method 1100 may be combined with one or more operations of method 1200. Additionally, or alternatively, one or more operations described above with reference to FIGS. 1-6, 7A, 7B, 8, 9, and 10 may be combined with one or more operations of FIGS. 11 and 12, or a combination of FIGS. 11 and 12.

The above specification and examples provide a complete description of the structure and use of illustrative examples. Although certain aspects have been described above with a certain degree of particularity, or with reference to one or more individual examples, those skilled in the art could make numerous alterations to aspects of the present disclosure without departing from the scope of the present disclosure. As such, the various illustrative examples of the methods and systems are not intended to be limited to the particular forms disclosed. Rather, they include all modifications and alternatives falling within the scope of the claims, and implementations other than the ones shown may include some or all of the features of the depicted examples. For example, elements may be omitted or combined as a unitary structure, connections may be substituted, or both. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one example or may relate to several examples. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively.

1. A therapy system comprising:
an oxygen source configured to provide oxygen;
a hemoglobin source configured to provide topical hemoglobin;
a mixer including a first inlet, a second inlet, and an outlet, the mixer configured to mix the oxygen and the topical hemoglobin to form a mixture and to provide the mixture to a dressing via the outlet; and
the dressing.

2. The therapy system of claim 1, wherein the oxygen source is a hydrolysis device configured to generate the oxygen, and wherein the topical hemoglobin is aerosol hemoglobin.

3. The therapy system of claim 1, wherein the topical hemoglobin is natural hemoglobin, synthetic hemoglobin, or a mixture thereof.

4. The therapy system of claim 1, wherein the topical hemoglobin is carbonylated hemoglobin.

5. The therapy system of claim 1, wherein the topical hemoglobin is fetal hemoglobin, adult hemoglobin, or a mixture thereof.

6. The therapy system of claim 1, wherein the oxygen is medically pure humidified oxygen.

7. The therapy system of claim 1, wherein the mixer comprises one of:
a spray nozzle to inject one of the oxygen or the topical hemoglobin into the other of the oxygen or the topical hemoglobin to form a homogenous mixture;
an in-line mixer configured to generate a homogenous mixture; or
a porous structure configured to mix the oxygen and the topical hemoglobin to form a homogenous mixture.

8. The therapy system of claim 1, further comprising tubing and a controller configured to control delivery of the oxygen, the topical hemoglobin, the mixture, or a combination thereof, to the dressing.

9. The therapy system of claim 1, further comprising one or more sensors.

10. A wound dressing comprising:
an absorbent material; and
a mixer coupled to the absorbent material and including an oxygen inlet, a hemoglobin inlet, and an outlet, the mixer configured to mix oxygen and hemoglobin to form a mixture and to provide the mixture to the absorbent material via the outlet.

11. The wound dressing of claim 10, wherein the mixer comprises a spray nozzle configured to inject one of the oxygen or the topical hemoglobin into the other of the oxygen or the topical hemoglobin to form a homogenous mixture.

12. The wound dressing of claim 10, wherein the mixer comprises an in-line mixer configured to generate a homogenous mixture.

13. The wound dressing of claim 12, wherein the in-line mixer has a spiral or square shape conduit for mixing the oxygen and the hemoglobin.

14. The wound dressing of claim 10, wherein the mixer comprises porous structure configured to mix the oxygen and the topical hemoglobin to form a homogenous mixture.

15. The wound dressing of claim 14, wherein the porous structure comprises a porous polymeric exchange material.

16. The wound dressing of claim 10, further comprising a manifold coupled to the absorbent material and to the mixer.

17. The wound dressing of claim 16, wherein the manifold comprises polyether based polyurethane foam, and wherein the mixer provides the mixture to the absorbent material via the manifold.

18. The wound dressing of claim 10, wherein the absorbent material comprises a printed absorbent.

19. The wound dressing of claim 10, further comprising a pad and a drape.

20. The wound dressing of claim 10, further comprising a sensor.

21. A method comprising:
mixing oxygen and topical hemoglobin to form a mixture; and
applying the mixture to a wound dressing attached to a tissue site.

22. The method of claim 21, wherein applying the mixture corresponds to hyperbaric oxygen treatment, and further comprising applying negative pressure therapy after the hyperbaric oxygen treatment, wherein the hyperbaric oxygen treatment and the negative pressure therapy may be cycled.

23. The method of claim 21, further comprising generating the oxygen.

24. The method of claim 21, further comprising:
providing the oxygen from an oxygen source; and
providing the topical hemoglobin in aerosol form from a compressed hemoglobin source.

25. A method comprising:
providing pressurized wound therapy to a tissue site via a wound dressing; and
while providing the pressurized wound therapy, providing a mixture of oxygen and topical hemoglobin to the wound dressing.

26. The method of claim 25, further comprising:
providing the oxygen from an oxygen source;
providing the topical hemoglobin in aerosol form from a compressed hemoglobin source; and
mixing the oxygen and the topical hemoglobin to form the mixture.

27. The method of claim 26, further comprising generating the oxygen.

28. A kit for oxygen wound therapy, the kit comprising:
one or more wound dressings as in claim 10.

29. The kit of claim 28, further comprising an oxygen source, a hemoglobin source, a mixture of oxygen and hemoglobin, or a combination thereof.

30. A kit for oxygen wound therapy, the kit comprising:
an oxygen source configured to provide oxygen;
a hemoglobin source configured to provide topical hemoglobin; and
a mixer including a first inlet, a second inlet, and an outlet, the mixer configured to mix the oxygen and the topical hemoglobin to form a mixture and to provide the mixture to a dressing via the outlet.

31. The kit of claim 30, further comprising one or more dressings.

32. A therapy system comprising:
an oxygen source configured to provide oxygen;
a hemoglobin source configured to provide topical hemoglobin; and
means for mixing the oxygen and the topical hemoglobin to form a mixture and to provide the mixture to a dressing via the outlet.

33. The therapy system of claim 32, wherein the oxygen, the topical hemoglobin, or both are stored under pressure.

34. The therapy system of claim 32, further comprising:
a dressing coupled to the means for mixing; and
a pump coupled to the means for mixing and configured to transport the mixture to the dressing.

35. A therapy system comprising:
a wound dressing; and
a container coupled to the wound dressing, the configured to provide a mixture of oxygen and hemoglobin to the dressing.

36. The therapy system of claim 35, the container further comprising means for mixing oxygen and topical hemoglobin to form the mixture.

37. The therapy system of claim 35, wherein the container includes the mixture of oxygen and hemoglobin, and wherein the mixture is stored under pressure.

38. The therapy system of claim 35, further comprising a pump coupled to the container and configured to transport the mixture to the dressing.
